(19) United States
(12) Patent Application Publication
(10) Pub. No.: US 2023/0250380 A1
(43) Pub. Date: Aug. 10, 2023

(54) BIOPROCESSING SYSTEM AND TUBING AND COMPONENT MANAGEMENT APPARATUS FOR A BIOPROCESSING SYSTEM

(71) Applicant: GLOBAL LIFE SCIENCES SOLUTIONS USA LLC, MARLBOROUGH, MA (US)

(72) Inventors: ELIZABETH STEGNER, RYE, NH (US); KANDAKUMAR MURUGESAN, BANGALORE-KARNATAKA (IN); SAIRAM CHERUNNI, BANGALORE-KARNATAKA (IN); MANAS DASH, WAYLAND, MA (US); PRASAD BAGIYANATHAN, BANGALORE-KARNATAKA (IN); SHIVAKUMAR SELVARAJ, BANGALORE-KARNATAKA (IN); BINEESH KANDOTH, BANGALORE-KARNATAKA (IN); NITYANANDA MAIYA, BANGALORE-KARNATAKA (IN); NICHOLAS BROCATO, MARLBOROUGH, MA (US); CHANDRA PRAKASH DHANARIYA, BANGALORE-KARNATAKA (IN); SURESH SUNDARAMOORTHY, BANGALORE-KARNATAKA (IN)

(73) Assignee: GLOBAL LIFE SCIENCES SOLUTIONS USA LLC, MARLBOROUGH, MA (US)

(21) Appl. No.: 18/014,370

(22) PCT Filed: Jul. 8, 2021
(86) PCT No.: PCT/EP2021/068988

§ 371 Date: Jan. 4, 2023

(30) Foreign Application Priority Data
Jul. 15, 2020 (IN) 202011030099

(51) Int. Cl. C12M 3/00; C12M 1/00; C12M 1/12

(52) U.S. Cl. CPC C12M 23/46; C12M 23/14; C12M 23/48; C12M 25/10; C12M 27/20

(57) ABSTRACT

A component management apparatus for a bioprocessing system includes a frame having a plurality of segments, including at least a first segment and a second segment pivotably connected to the first segment such that at least the second segment is movable between a closed position and an open position, and at least one mounting bracket connected to the frame for connection of a bioprocess component.

### BACKGROUND

### Technical Field

Embodiments of the invention relate generally to bioprocessing systems and methods and, more particularly, to a tubing and component management system for a bioprocessing system.

### DISCUSSION OF ART

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless steel shell or vessel. Use of sterilized disposable bags eliminates the time-consuming step of cleaning of the vessel and reduces the chance of contamination. The bag may be positioned within the rigid vessel and filled with the desired fluid for mixing. Depending on the fluid being processed, the system may include a number of fluid lines and different sensors, probes and ports coupled with the bag for monitoring, analytics, sampling, and fluid transfer. For example, a plurality of ports may typically be located at the front of the bag and accessible through an opening in the sidewall of the vessel, which provide connection points for sensors, probes and/or fluid sampling lines. In addition, a harvest port or drain line fitting is typically located at the bottom of the disposable bag and is configured for insertion through an opening in the bottom of the vessel, allowing for a harvest line to be connected to the bag for harvesting and draining of the bag after the bioprocess is complete.

Typically, an agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel. Magnetic coupling of the agitator inside the bag, to a drive system or motor external to the bag and/or bioreactor vessel, can eliminate contamination issues, allow for a completely enclosed system, and prevent leakage. Because there is no need to have a drive shaft penetrate the bioreactor vessel wall to mechanically spin the agitator, magnetically coupled systems can also eliminate the need for having seals between the drive shaft and the vessel.

Installation and setup of the flexible bioprocessing bag within the bioreactor vessel, along with the associated tubing, filter heaters, valves, impeller and other components can be a labor intensive and time-consuming process. For example, existing bioreactor vessels may present accessibility issues, making it difficult to align and properly seat the impeller with the bioreactor vessel base. Multiple operators and ladders may also be needed, especially for the installation of tubing and filter heaters, which are located at the top of the vessel. Moreover, lack of tubing support for the various tubes connected to the flexible bag can lead to a cluttered array of tubes around the bioreactor vessel.

In view of the above, there is a need for a tubing and component management system for a bioprocessing system that is modular, ergonomically efficient and facilitates installation and setup.

### BRIEF DESCRIPTION

In an embodiment, a component management apparatus for a bioprocessing system includes a frame having a plurality of segments, including at least a first segment and a second segment pivotably connected to the first segment such that at least the second segment is movable between a closed position and an open position, and at least one mounting bracket connected to the frame for connection of a bioprocess component.

In another embodiment, a bioprocessing system includes a vessel defining an interior space for receiving a flexible bioprocessing bag, the vessel having an access door in a sidewall of the vessel providing access to the interior space, and a tubing and component management apparatus operatively connected to the vessel for mounting at least one component of the bioprocessing system. The tubing and component management apparatus includes a frame having a plurality of segments including at least a first segment and a second segment pivotably connected to the first segment such that at least the second segment is movable between a closed position and an open position, and at least one mounting bracket connected to the frame for connection of the at least one component.

In yet another embodiment, a filter heater assembly for a bioprocessing system includes a first casing portion and a second casing portion hingedly connected to the second casing portion, wherein the second casing portion is movable between a closed position in which the filter heater receives a filter, and an open position enabling installation or removal of the filter.

In yet another embodiment, a filter holder assembly includes a sleeve having a plurality of interconnected sleeve elements, the sleeve having a top opening, wherein the sleeve has a cross-sectional area that is greatest at the top opening and decreases moving away from the top opening, and wherein the sleeve elements are configured so as to be biased outwardly upon insertion of a filter heater through the top opening.

### DRAWINGS

The present invention will be better understood from reading the following description of non-limiting embodiments, with reference to the attached drawings, wherein below:

FIG. 1 is a perspective view of a bioprocessing system according to an embodiment of the invention.

FIG. 2 is a perspective view of a component management apparatus of the bioprocessing system of FIG. 1, according to an embodiment of the present invention.

FIG. 3 is an enlarged, perspective view a portion of the component management apparatus of FIG. 2.

FIG. 4 is a perspective view of the component management apparatus of FIG. 2, shown with various components installed.

FIG. 5 is a perspective view of a component management apparatus according to another embodiment of the present invention.

FIG. 6 is a top plan view of the component management apparatus of FIG. 5.

FIG. 7 is a side elevational view of the component management apparatus of FIG. 5.

FIG. 8 is a perspective view of a filter heater according to an embodiment of the present invention, showing an open position of the casing.

FIG. 9 is a perspective view of the filter heater of FIG. 8, showing a closed position of the casing.

FIG. 10 is a perspective view of the filter heater of FIG. 8, shown with a thermal jacket received around the casing.

FIG. 11 is a perspective view of a filter heater according to another embodiment of the present invention, showing an open position of the casing.

FIG. 12 is a perspective view of the filter heater of FIG. 11, showing a filter received in the casing.

FIG. 13 is a perspective view of the filter heater of FIG. 11, showing a closed position of the casing.

FIG. 14 is a perspective view of a mounting bracket of the filter heater of FIG. 11.

FIG. 15 is a perspective view of a filter heater having a mechanical locking mechanism, according to an embodiment of the invention.

FIG. 16 is a perspective view of a filter heater having a magnetic locking mechanism, according to an embodiment of the invention.

FIG. 17 is a perspective view of a filter heater holder, according to an embodiment of the invention.

FIG. 18 is a side elevational view of the filter heater holder of FIG. 17.

FIG. 19 is a top plan view of the filter heater holder of FIG. 17.

FIG. 20 is a perspective view of the filter heater holder of FIG. 17, illustrating use of the filter heater holder with filter heaters of various sizes.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems, e.g., chromatography and tangential flow filter systems, and their associated flow paths. As used herein, the term “bag” means a flexible or semi-rigid container or vessel used, for example, as a bioreactor or mixer for the contents within. As used herein, “consumable” or “consumable component” means devices or components that are intended to be replaced regularly due to wear or use.

Embodiments of the invention provide bioprocessing systems and, in particular, component management systems and devices for a bioreactor system. In an embodiment, a bioprocessing system includes a vessel defining an interior space for receiving a flexible bioprocessing bag, the vessel having an access door in a sidewall of the vessel and providing access to the interior space, and a component management apparatus mounted to the sidewall of the vessel and having a mounting frame for mounting of at least one consumable component of the bioprocessing system. The mounting frame is moveable vertically into and out of the interior space.

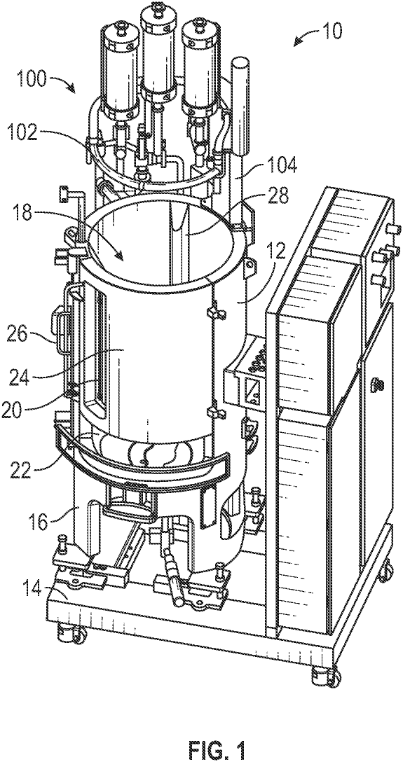

With reference to FIG. 1, a bioprocessing system 10 (also referred to herein as bioreactor system 10) according to an embodiment of the invention is illustrated. The bioreactor system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 can be any shape or size as long as it is capable of supporting a single-use, flexible bioreactor bag in an interior space 18 thereof. For example, according to one embodiment of the invention the vessel 12 is capable of accepting and supporting a 10L-2000L flexible or collapsible bioprocess bag.

The vessel 12 may include one or more sight windows 20, which allows an operator to view a fluid level within the flexible bag positioned within the interior space 18, as well as a window 22 positioned at a lower area of the vessel 12. The window 22 allows access to the interior of the vessel 12 for insertion and positioning of various sensors and probes (not shown) within the flexible bag, and for connecting one or more fluid lines to the flexible bag for fluids, gases, and the like, to be added or withdrawn from the flexible bag. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

In an embodiment, the vessel 12 includes an access door 24 hingedly or pivotally connected to a sidewall of the vessel 12 permitting access to the interior space 18. The door 24 may include a handle 26 that facilitates movement of the door between the open and closed positions. In an embodiment, the door 24 may be configured and positioned such that when the door 24 is in the closed position, a lower edge of the door 24 forms an upper edge or boundary of the window 22, and/or a side edge of the door 24 forms an edge or boundary of the window 20. By having the edges of the door 24 define one or more boundaries of the windows 22, 24, when the door 22 is in the open position, a contiguous and unobstructed access opening in the sidewall of the vessel is formed by the opening 20, opening 22 and open door 24 (i.e., the opening in which the door is received). Accordingly, the area of the contiguous access opening formed in the sidewall of the vessel 12 when the door is in the open position is equivalent to the combined areas of the door 24, window 22 and window 24. This provides greater clearance and access to the interior space 18 than would otherwise be possible if the door and windows were separated by a portion of the sidewall of the vessel 12.

With further reference to FIG. 1, the interior sidewall of the vessel 12 may include one or more vertical baffles 28 that project into the interior space 18. The baffles 28 may be generally triangular in cross-section, although shapes and configurations known in the art may also be utilized without departing from the broader aspects of the invention. The baffles 28 are configured to contact and bias the flexible bag (when installed in the interior space 18) inwardly during a bioprocessing operation, for purposes known in the art.

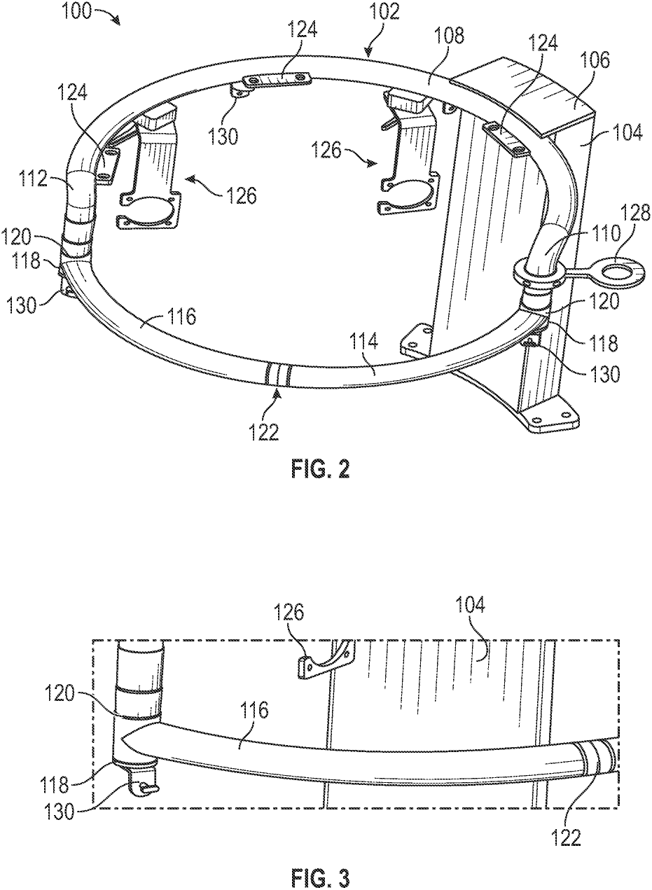

As further shown in FIG. 1, the bioreactor system 10 also includes a component management apparatus 100. The apparatus 100 includes a frame 102 connected to a vertically-oriented support 104 via top plate 106, as illustrated in FIG. 2. The support 104 is operatively connected to the vessel 12 in a manner such that the support 104, and the frame 102 carried thereon, is vertically moveable with respect to the vessel 12 so that the frame 102 can be moved vertically into and out of the interior space 18, also known as a “floating frame”. It is envisioned that various moving mechanisms/devices may be utilized for raising and lowering the support 104 and frame 102, and it is not intended that the invention be limited to any particular configuration. In an embodiment, the moving mechanism may be, for example, a hand-driven mechanism like a cable and hand crank, or an actuator such as an electric motor. According to an alternative embodiment, the apparatus 100 may have a set height above the vessel, also known as a “fixed frame”. With a fixed frame configuration, no moving mechanisms/devices are required. Additionally, and with regard to the fixed frame embodiment, there may be additional support structures 104 for providing support to the frame 102 in its attachment to the vessel 12. For example, additional support structures 104 may extend or attach to portions the frame 102 (e.g., below the locations of bushings 118) that attach to the vessel 12. Further, the flexible bag may be installed such that it is hoisted into a working position via the aforementioned moving mechanisms/devices (i.e., the frame is configured to lift the flexible bag into its working position). Alternatively, the flexible bag may be hung directed onto the frame when it is located above the vessel 12. Still further, a separate moving mechanism/device (e.g., a hoist) can be configured to lift flexible bag into its working position.

Turning now to FIGS. 2 and 3, detailed views of the component management apparatus 100 are shown. As illustrated therein, the frame 102 is generally annular in shape and includes a plurality of interconnected segments. For example, in an embodiment, the frame 102 includes a first segment 108 that is arcuate in shape and has opposed distal ends 110, 112, a second segment 114 pivotably connected to the first end 110 of the first segment 108, and a third segment 116 pivotably connected to the second end 112 of the first segment 108. As illustrated in FIG. 2, the distal ends 110, 112 of the first segment 108 may, in an embodiment, have a generally 90 degree bend which facilitates connection of the second and third segments 114, 116 to the respective ends of the first segment 108. In this respect, the second and third segments 114, 116 lie in a plane that is vertically spaced from a plan in which the first segment 108 lies. In an embodiment, bushings 118 and nylon 120 washers may be utilized to facilitate rotational or pivoting movement of the second and third segments 114, 116 with respect to the first segment 108. It is envisioned that the frame 102 may be manufactured from a variety of materials known in the art such as, for example, stainless steel.

As discussed in detail below, the second and third segments 114, 116 are pivotable with respect to the first segment 108 such that they may be positioned in a closed position (shown in FIG. 2), where the segments, together, form a closed ring, and an open position allowing access to the space interior to the frame 102. As further shown in FIG. 2, the second and third segments 114, 116 are shaped and dimensioned so that distal ends of the second and third segments 114, 116, respectively, are in close association with one another when in the closed position. In an embodiment, the distal ends of the second and third segments 114, 116 are configured with a complimentary connection mechanism 122 configured to maintain the second and/or third segment in the closed position. In an embodiment the connection mechanism 122 is a mechanical lock. In another embodiment, the connection mechanism 122 is a magnetic coupling. As indicated above, the second and third segments 114, 116 can be placed in the closed position and held in such position by the connection mechanism 122. When it is desired to move the second and/or third segment 114, 116 to the open position, a user can unlock the connection mechanism or rotate the segment(s) to break the magnetic attraction of the connection mechanism, as the case may be.

With further reference to FIG. 2, the frame 102 is outfitted with a plurality of mounting brackets that enable various bioreactor components to be connected to the frame 102. For example, the mounting brackets may include a plurality of filter heater mounting brackets 124 for connecting filter heaters to the frame 102 and a plurality of pinch valve mounting brackets 126 for connecting pinch valves to the frame. In an embodiment, the filter heater mounting brackets 124 may be welded or bolted to the frame 102. In yet other embodiments, the brackets 124 may be connected to the frame via a bracket so that the brackets 124 can be easily added and positioned as desired. As best shown in FIG. 2, pinch valve mounting brackets 126 are mounted to the underside of the frame 102 and depend downwardly therefrom. In an embodiment, the mounting brackets 126 are rotatable about a vertical axis and include a knob (not shown) that can be used to adjust the angle of the pinch valve (when received by the mounting bracket 126) so that a pinch valve can be used with one filter heater or rotated and used with a different filter heater.

The frame 102 may also include a ring-shaped mounting bracket 128 connected to the frame 102, such as by welding. The mounting bracket 128 is specifically designed to accommodate a plurality of stacklights used to provide a visual indication of certain bioprocess conditions. Still further, the frame 102 includes a plurality of hanger brackets 130 equally spaced around the frame 102. The hanger brackets 130 are utilized as mounting points for suspending the flexible bioprocessing bag. In particular, the bioprocessing bag has a plurality of suspension points spaced around the top of the bag, with a grommet associated with each suspension point. A hanger strap is connected to each hanger bracket 130, and the carabiner is then attached to the grommet to suspend the flexible bag from the frame 102.

In use, the frame 102 may be lowered into the bioreactor vessel 12 using the moving mechanism associated with the support 104. Once positioned within the interior space 18, the door 24 may be opened. At this point, the second and third segments 114, 116 may be rotated to their respective open positions where they may partially extend from the opening in the sidewall of the vessel 12. In this position, all points on the frame are ergonomically accessible, enabling a user to easily and efficiently mount all necessary components for a bioprocessing operation (and to disassemble, as necessary, after use), and to organize all lines and cables. For example, filter heaters, pinch valves, pressure sensors, as well as the cables and tubes required to operate them, and liquid and gas lines can be easily mounted to the frame using the mounting brackets. Once all components are mounted to the frame 102, the frame segments may be moved to their respective closed positions and the frame 102 may be raised to an operational position.

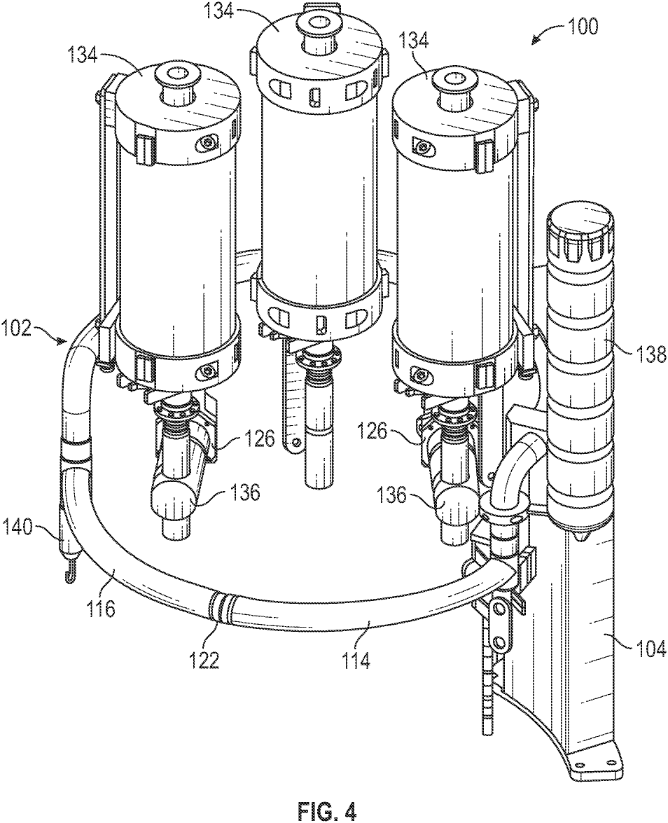

FIG. 4 illustrates filter heaters 134 mounted to the mounting brackets 124, pinch valves 136 mounted to the mounting brackets 126, and stacklights 138 mounted to the mounting bracket 128. FIG. 4 also shows the hanger straps 140 connected to the hanger brackets 130.

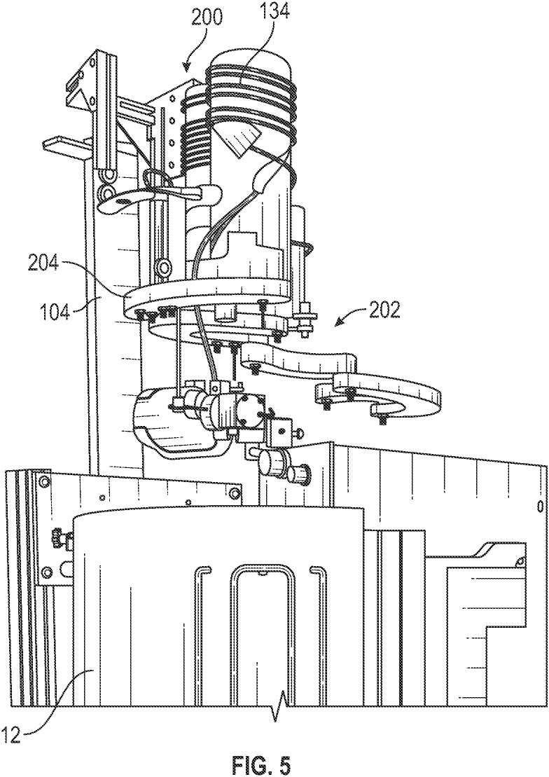

Turning now to FIGS. 5-7, a component management apparatus 200 according to another embodiment of the invention is illustrated. The component management apparatus 200 is generally similar in configuration and operation to component management apparatus 100 described above. In particular, component management apparatus 200 includes a frame 202 having a plurality of rotationally or pivotally interconnected segments which, in a closed position, define a peripheral shape of the frame 202 that generally corresponds to the shape of the interior of the bioreactor vessel 12 (including the sidewalls and baffles).

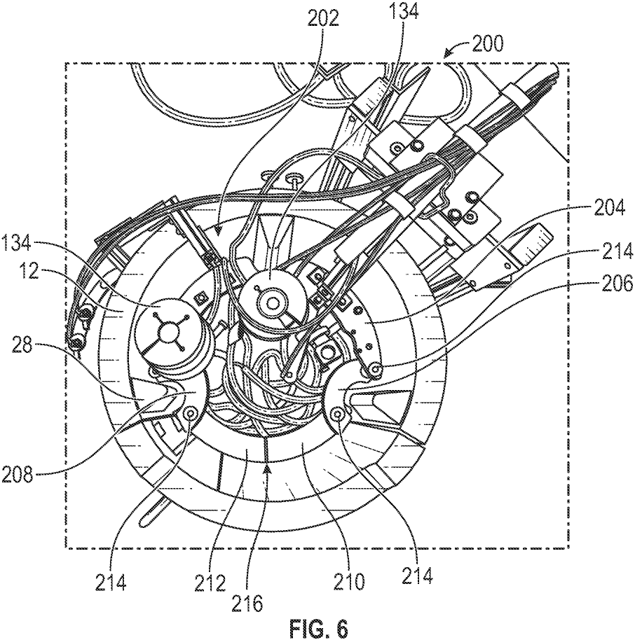

In particular, as best shown in FIG. 6, the frame 202 includes, at least, a first segment 204 having a peripheral shape and radius that generally corresponds to the shape and radius of the interior of the bioreactor vessel 12, second and third segments 206, 208 pivotally connected to opposed ends of the first segment 204, and fifth and sixth segments 210, 212 that are pivotally connected to the second and third segments 206, 208, respectively. The segments are each rotatable about a vertical axis defined by pivot points 214 where the segments connect to one another. Similar to the embodiment described above, the endmost segments which complete the annulus are configured with a connection mechanism, e.g., a magnetic coupling 216, that retains the frame 202 in the closed position.

As further shown in FIG. 6, while segments 204, 210 and 212 have a generally convex outer periphery, segments 206 and 208 have a generally concave outer periphery, which enables the frame 202 to be placed in close associate with the interior sidewalls of the bioreactor vessel 12, even in the case where internal baffles 28 are present. In particular, the segments 206, 208 are shaped and dimensioned so as to accommodate internal baffles 28 of the bioreactor vessel. In other words, the segments, as a whole, are shaped and dimensioned to closely correspond to the shape of the interior walls of the bioreactor vessel (including the baffles).

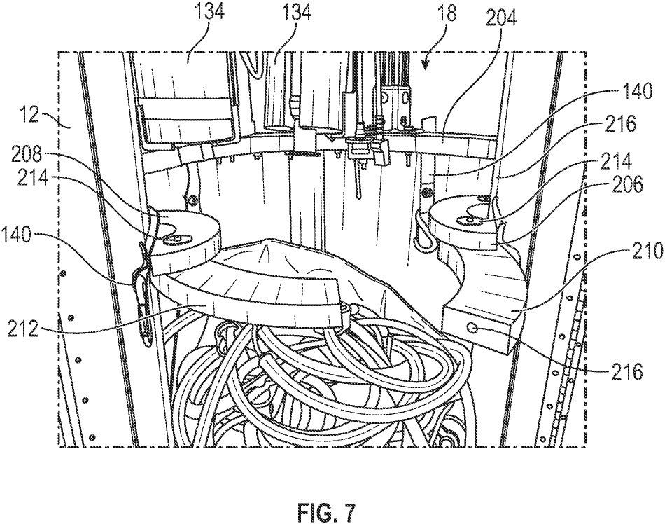

As best shown in FIG. 7, the second and third segments 206, 208 are interconnected with the first segment 204 via vertical posts or axles 216 at the pivot points 214, which function to vertically space the second and third segments 206, 208 from the first segment 204 (i.e., the second and third segments 206, 208 are at vertically lower location than the first segment 204). Similarly, the fourth and fifth segments 210, 212 are connected to the second and third segments 206, 208 such that they are positioned vertically lower than the second and third segments 206, 208. As illustrated in FIG. 7, the fourth and fifth segments 210, 212 are rotatable about their respective pivot points 214 against the holding force of the connection mechanism 216 to a position where they extend at least partially through the access door opening in the sidewall of the bioreactor vessel 12. This configuration, therefore, allows ergonomic access to in entire frame 202, facilitating the easy connection of components like filter heaters, filters, pinch valves, stacklights, sensors and tubing.

In connection with the above, while not fully illustrated in FIG. 7, the frame 202 may be outfitted with an array of mounting brackets, e.g., filter heater mounting brackets, pinch valve mounting brackets, stacklight mounting brackets, bag hangers, and the like, of the type more particularly described above in connection with the component management apparatus 100.

As disclosed above, the component management apparatuses of the invention include a frame that supports all the necessary bioreactor components at the top of the bioreactor, including filter heaters, pinch valves, pressure sensors, and liquid and gas lines. The frame enables intuitive, ergonomic installation of all components, and facilitates organization of all lines and cables. The frame fits within the interior space 18 of the bioreactor 12 and can be lowered into the bioreactor vessel or remain at the maximum height. The apparatuses 100, 200 are modular to enable users to add or remove features as necessary (for instance, if a user needed 4 filter heaters instead of 3, he/she could easily add in a feature to hold the extra filter heater). Because the frame is hinged, it maintains a very small footprint but can expand for optimum ease of use. In addition, the configuration of the frame enables users to make all gas line and electrical connections quickly and easily, while maintaining appropriate biomechanics.

In connection with the above, and as will be appreciated, single use bioreactor bags have a mess of tubes at top that are difficult to deal with. The component management apparatuses disclosed herein support the tubes and other components without blocking a user from reaching other necessary components. Moreover, because the frame is in the shape of an annulus corresponding generally to the shape of the bag and bioreactor vessel, users are able to route the tubes 360°. The articulating segments of the frame enable a user to open the frame and access to the tubes directly, as well as reach farther into the vessel uninhibited to make other connections, providing an ease of installation, disassembly and use heretofore not seen in the art.

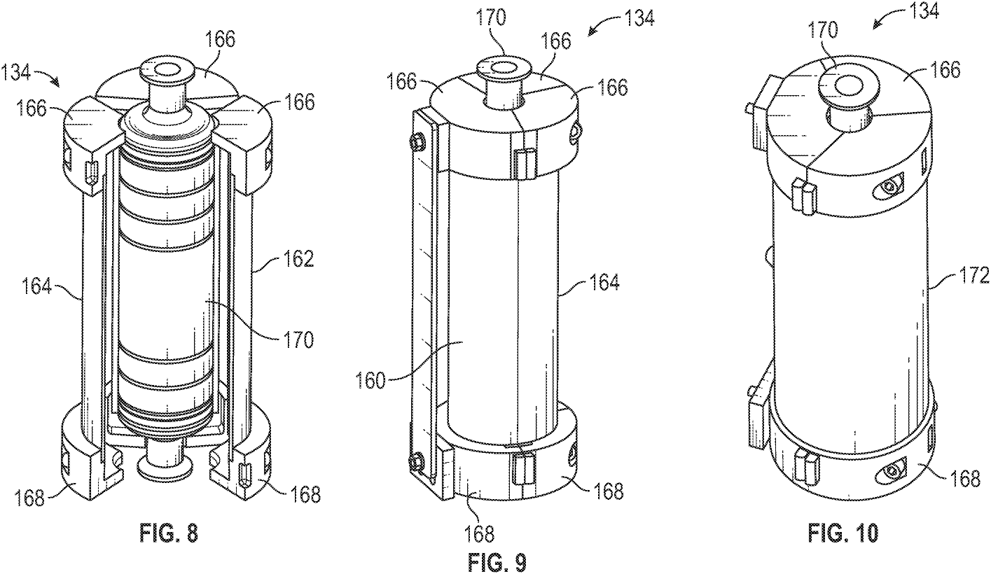

Referring now to FIGS. 8-10, detailed views of the filter heater 134 according to one embodiment of the invention are shown. The filter heater 134 is designed to provide uniform heat distribution to the exhaust gas passing through the filter positioned inside the filter heater 134. As shown in FIGS. 8-10, the filter heater 134 includes a first casing portion 160, a second casing portion 162 and a third casing portion 164, wherein each casing portion 160, 162, 164 includes a top cap member 166 and a bottom cap member 168. The vertical portion of the casing extending between the top cap member and bottom cap member of each casing is formed or configured as a heat distributor for distributing heat to a filter 170 removably received within the filter heater 134. As shown in FIG. 8, the second and third casing portions 162, 164 are hingedly connected to the first casing portion 162, 164 about respective vertical axes, allowing the filter heater 134 to be opened, allowing for insertion of the filter 170 into the filter heater 134. Once the filter 170 is inserted, the second and third casing portions 162, 164 can be closed to retain the filter 170 therein, as shown in FIG. 9. In an embodiment, the second and third casing portions 162, 164 are configured with a latching or locking mechanism such as, for example, a magnetic coupling, Velcro, snaps or the like, for maintaining the casing portions in the closed position. In an embodiment, the top and bottom cap members 166, 168 may be manufactured from a thermoplastic material such as, for example, polyetherimide, and the heat distributor portion of the casing may be formed from aluminum, although other materials capable of distributing heat to the filter 170 retained in the casing may also be utilized without departing from the broader aspects of the invention.

With particular reference to FIG. 10, the filter heater 134 further includes a thermal jacket or heating blanket 172 that is wrapped around the periphery of the filter heater 134. The thermal jacket 172 is configured to transfer heat to the casing, which is then transferred to the filter 170, heating the gases passing therethrough. In an embodiment the thermal jacket 172 may be retained on the filter heater 134 using straps, snaps or other fasting means. As indicated above, the filter heater 134 is mountable to the filter heater mounting brackets 124 on the frame of the component management apparatus 100, 200.

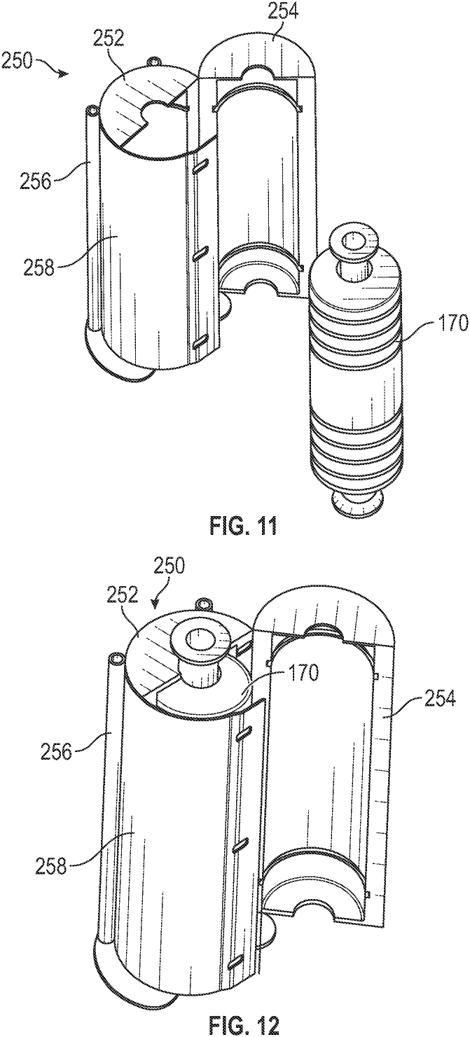

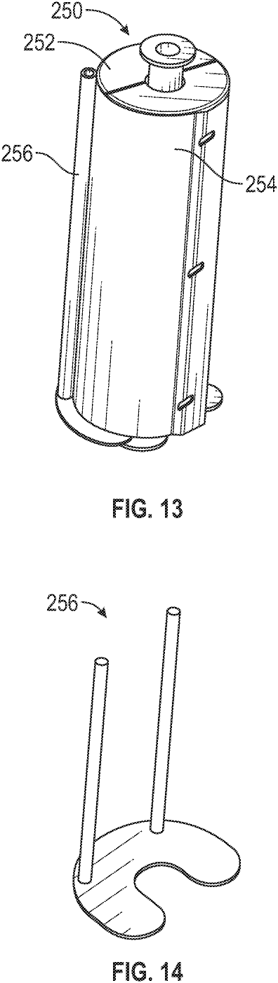

With reference to FIGS. 11-16 a filter heater 250 according to another embodiment of the invention is shown. The filter heater 250 is generally similar to the filter heater 134 disclosed above, however, rather having a three-piece casing, the filter heater 250 includes a two-piece casing. In particular, filter heater 250 includes a first casing portion 252 and a second casting portion 254 hingedly connected to the first casing portion 252 about a vertical axis. The filter heater 250 further includes a bracket 256 attached to the first casing portion 252 enabling mounting of the filter heater 250 to the corresponding mounting bracket on the frame of the component management apparatus. As shown in FIG. 11, the second casing portion 254 is pivotable to an open position, enabling a filter 170 to installed and retained by the first casing portion 252 without slipping. Once received by the first casing portion, as shown in FIG. 12, the second casing portion 254 may be closed, as shown in FIG. 13. The filter heater 250 also includes a thermal jacket or blanket 258 that wraps around the casings 252, 254 for transferring heat to the casings 252, 254 and, ultimately, to the filter 170 retained thereby.

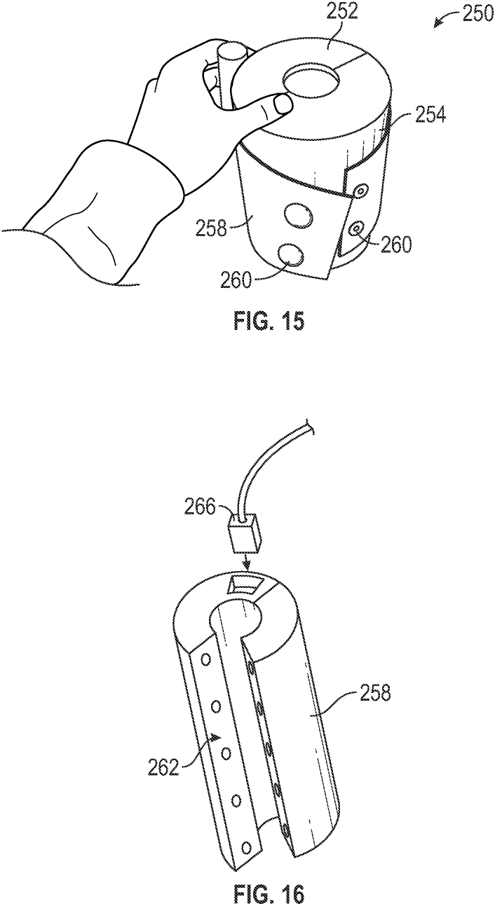

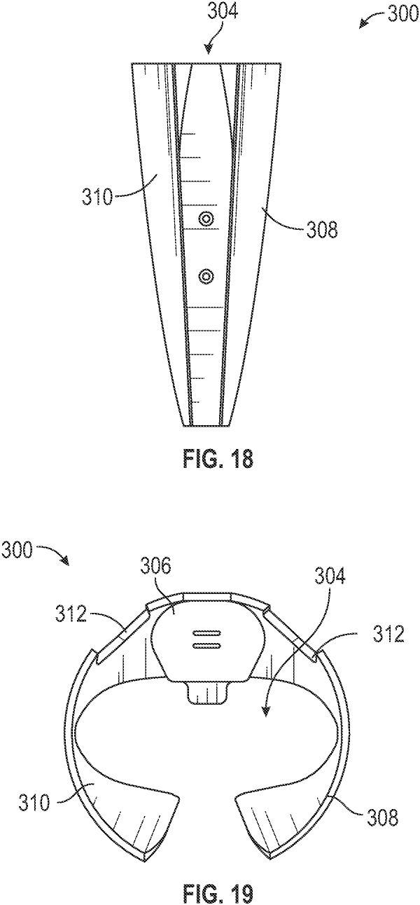

With specific reference to FIG. 15, in an embodiment, the thermal jacket 258 may be retained on the filter heater 250 using snaps 260, however, other fastening means such as Velcro, magnets or the like may also be utilized without departing from the broader aspects of the invention. For example, as shown in FIG. 16, the thermal jacket 258 may be retained on the filter heater using a magnetic coupling 264. As also shown therein, in an embodiment, the thermal jacket 258 may be powered by a removable cable 266. The removable cable 266 allows for easy internal routing (installation and disassembly) of the cable 266. It is to be noted that the thermal jacket 258, and its associated fastening means may equally be implemented with the filter heaters of the embodiments of FIGS. 8-10 and 17-20.

The filter heaters disclosed herein provide an increased level of accessibility for inserting and removing the filter, and ensures that the filter does not slip from the filter holder. This reduces the assembly time of the filter and improves accessibility of the system, as a whole.

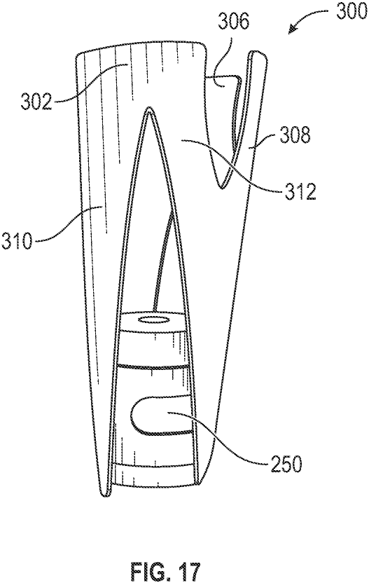

Turning now to FIGS. 17-19, a filter heater holder 300 that may be used with the bioprocessing system 100 is illustrated. As is known in the art, filters are extensively utilized in bioreactors and come in various diameters and heights. As discussed above, these filters are enveloped by a filter heater. Current filter holders are only capable of accommodating a particular size filter, which limits the ability of a customer to use different size filters. As shown in FIGS. 17-19 the filter heater holder 300 of the invention includes a sleeve 302 having a top opening 304. The sleeve 302 is formed by a plurality sleeve elements (e.g., sleeve elements 306, 308, 310) that are interconnected with one another by webs 312 of material adjacent to the top opening 304. In an embodiment, the sleeve 302 has a cross-sectional area that is greatest at the top opening 304 and decreases moving away from the top opening 304. For example, the sleeve 302 may be frusto-conical in shape. The sleeve elements 306, 308, 310 are configured and joined together via the webs 312 such that the distal portions of the sleeve elements opposite the top opening can be resiliently biased by a filter heater, e.g., filter heater 250, when inserted through the top opening 304.

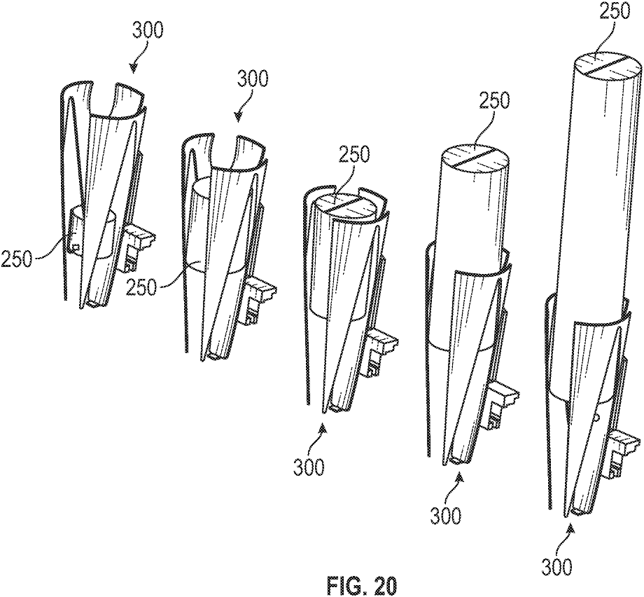

As shown in FIG. 20, this configuration allows the filter heater holder 300 to accommodate filter heaters 250 of various sizes (e.g., diameters and heights). In particular, as the filter heater 250 is urged downwardly through the opening 304, the distal portions of the sleeve elements are biased outwardly, allowing the filter heater 250 to be received by the holder 300. The filter heater holder 300 may be connected to the frame of the component management apparatus using brackets or other hardware, e.g., mounting brackets 124.

Each of the filter heater holder embodiments discussed above are configured to connection to the component management apparatus 100, 200. While not explicitly shown in the figures, combinations of such filter heater holders may be implemented (e.g., attached to) the component management apparatus 100, 200.

The invention disclosed herein, as a whole, therefore is modular, ergonomically efficient and facilitates installation and setup of bioreactor components (and, in particular, consumable components) to a level heretofore not seen in the art.

In an embodiment, a component management apparatus for a bioprocessing system is provided. The apparatus includes a frame having a plurality of segments, including at least a first segment and a second segment pivotably connected to the first segment such that at least the second segment is movable between a closed position and an open position, and at least one mounting bracket connected to the frame for connection of a bioprocess component. In an embodiment, the apparatus may also include a connection mechanism configured to maintain the second segment in the closed position. In an embodiment, the connection mechanism may be a mechanical lock or magnetic coupling. In an embodiment, the at least one mounting bracket is a filter heater mounting bracket configured for mounting a filter heater in a position generally above the frame. In an embodiment, the at least one mounting bracket is a pinch valve mounting bracket configured for receiving a pinch valve, the pinch valve mounting bracket depending downwardly from the frame. In an embodiment, the at least one mounting bracket is a plurality of mounting brackets, wherein the plurality of mounting brackets include a plurality of filter heater mounting brackets configured for mounting filter heaters in a position generally above the frame, and wherein the plurality of mounting brackets further include a plurality of pinch valve mounting brackets configured for receiving pinch valves, the plurality of pinch valve mounting brackets depending downwardly from the frame. In an embodiment, the plurality of segments further include at least a third segment, wherein the first segment is arcuate in shape and has a first end and a second end, wherein the second segment is pivotably connected to the first end of the first segment, and wherein the third segment is pivotably connected to the second end of the first segment. In an embodiment, at least one of the first segment, the second segment and/or the third segment is shaped and/or dimensioned to correspond to an internal wall of a bioreactor vessel, and at least another of the first segment, the second segment and/or the third segment is shaped and dimensioned so as to accommodate an internal baffle of the bioreactor vessel. In an embodiment, the frame has a peripheral shape that corresponds to an interior sidewall of the bioreactor vessel and internal baffles of the bioreactor vessel. In an embodiment, at least one of the segments is movable so as to extend from an opening in a bioreactor vessel to which the frame is connected. In an embodiment, the apparatus also includes a filter heater mounted to the filter heater mounting bracket, the filter heater including at least a first casing portion and a second casing portion hingedly connected to the second casing portion, wherein the second casing portion is movable between a closed position in which the filter heater receives a filter, and an open position enabling installation or removal of the filter. In an embodiment, the apparatus further includes a filter heater holder mounted to the filter heater mounting bracket, the filter heater holder including a plurality of interconnected sleeve elements forming a sleeve having a top opening, the sleeve having a cross-sectional area that is greatest at the top opening and decreases moving away from the top opening, wherein the sleeve elements are configured so as to be biased outwardly upon insertion of a filter heater or filter through the top opening.

In another embodiment, a bioprocessing system is provided. The bioprocessing system includes a vessel defining an interior space for receiving a flexible bioprocessing bag, the vessel having an access door in a sidewall of the vessel providing access to the interior space, and a tubing and component management apparatus operatively connected to the vessel for mounting at least one component of the bioprocessing system. The tubing and component management apparatus includes a frame having a plurality of segments including at least a first segment and a second segment pivotably connected to the first segment such that at least the second segment is movable between a closed position and an open position, and at least one mounting bracket connected to the frame for connection of the at least one component. In an embodiment, the frame is moveable vertically into and out of the interior space. In an embodiment, the tubing and component management apparatus further includes a connection mechanism configured to maintain the second segment in the closed position, wherein the connection mechanism is a mechanical lock or magnetic coupling. In an embodiment, the at least one mounting bracket is a plurality of mounting brackets, wherein the plurality of mounting brackets include a plurality of filter heater mounting brackets configured for mounting filter heaters in a position generally above the frame, and wherein the plurality of mounting brackets further include a plurality of pinch valve mounting brackets configured for receiving pinch valves, the plurality of pinch valve mounting brackets depending downwardly from the frame. In an embodiment, the frame has a peripheral shape that corresponds to an interior sidewall of the bioreactor vessel and internal baffles of the bioreactor vessel. In an embodiment, at least one of the segments is movable so as to extend from an access door opening in the bioreactor vessel. In an embodiment, the bioprocessing system may include a filter heater having a first casing portion and a second casing portion hingedly connected to the second casing portion, wherein the second casing portion is movable between a closed position in which the filter heater receives a filter, and an open position enabling installation or removal of the filter. In an embodiment, the first casing portion and the second casing portion each include a top cap member and a bottom cap member, and a heat distributor extending between the top cap member and the bottom cap member. In an embodiment, the heat distributor is formed from anodized aluminum. In an embodiment, the filter heater assembly further includes a magnetic coupling configured to retain the second casing portion in the closed position. In an embodiment, the filter heater further includes a heating blanket receivable about the casing. In an embodiment, the heating blanket includes a mechanical coupling or magnetic coupling for retaining the heating blanket one the casing. In an embodiment, the bioprocessing system may also include a filter holder assembly having a sleeve having a plurality of interconnected sleeve elements and a top opening, wherein the sleeve has a cross-sectional area that is greatest at the top opening and decreases moving away from the top opening, and wherein the sleeve elements are configured so as to be biased outwardly upon insertion of a filter heater through the top opening. In an embodiment, the plurality of sleeve elements are interconnected by webs of material adjacent to the top opening, wherein distal portions of the plurality of sleeve elements opposite the top opening are configured to be resiliently biased by the filter heater.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A component management apparatus for a bioprocessing system, comprising:
a frame having a plurality of segments, including at least a first segment and a second segment pivotably connected to the first segment such that at least the second segment is movable between a closed position and an open position; and
at least one mounting bracket connected to the frame for connection of a bioprocess component.

2. The component management apparatus of claim 1, further comprising:
a connection mechanism configured to maintain the second segment in the closed position.

3. The component management apparatus of claim 2, wherein:
the connection mechanism is a mechanical lock or magnetic coupling.

4. The component management apparatus of claim 1, wherein:
the at least one mounting bracket is a filter heater mounting bracket configured for mounting a filter heater in a position generally above the frame.

5. The component management apparatus of claim 1, wherein:
the at least one mounting bracket is a pinch valve mounting bracket configured for receiving a pinch valve, the pinch valve mounting bracket depending downwardly from the frame.

6. The component management apparatus of claim 1, wherein:
the at least one mounting bracket is a plurality of mounting brackets;
wherein the plurality of mounting brackets include a plurality of filter heater mounting brackets configured for mounting filter heaters in a position generally above the frame; and
wherein the plurality of mounting brackets further include a plurality of pinch valve mounting brackets configured for receiving pinch valves, the plurality of pinch valve mounting brackets depending downwardly from the frame.

7. The component management apparatus of claim 1, wherein:
the plurality of segments further include at least a third segment;
wherein the first segment is arcuate in shape and has a first end and a second end;
wherein the second segment is pivotably connected to the first end of the first segment; and
wherein the third segment is pivotably connected to the second end of the first segment.

8. The component management apparatus of claim 7, wherein:
at least one of the first segment, the second segment and/or the third segment is shaped and/or dimensioned to correspond to an internal wall of a bioreactor vessel; and
at least another of the first segment, the second segment and / or the third segment is shaped and dimensioned so as to accommodate an internal baffle of the bioreactor vessel.

9. The component management apparatus of claim 1, wherein:
the frame has a peripheral shape that corresponds to an interior sidewall of the bioreactor vessel and internal baffles of the bioreactor vessel.

10. The component management apparatus of claim 1, wherein:
at least one of the segments is movable so as to extend from an opening in a bioreactor vessel to which the frame is connected.

11. The component management apparatus of claim 4, further comprising:
a filter heater holder mounted to the filter heater mounting bracket, the filter heater holder including a plurality of interconnected sleeve elements forming a sleeve having a top opening, the sleeve having a cross-sectional area that is greatest at the top opening and decreases moving away from the top opening;
wherein the sleeve elements are configured so as to be biased outwardly upon insertion of a filter heater or filter through the top opening.

12. A bioprocessing system, comprising:
a vessel defining an interior space for receiving a flexible bioprocessing bag, the vessel having an access door in a sidewall of the vessel providing access to the interior space; and
a tubing and component management apparatus operatively connected to the vessel for mounting at least one component of the bioprocessing system;
wherein the tubing and component management apparatus includes a frame having a plurality of segments including at least a first segment and a second segment pivotably connected to the first segment such that at least the second segment is movable between a closed position and an open position, and at least one mounting bracket connected to the frame for connection of the at least one component.

13. The bioprocessing system of claim 12, wherein:
the frame is moveable vertically into and out of the interior space.

14. The bioprocessing system of claim 12, wherein:
the frame is fixed to the vessel.

15. The bioprocessing system of any one of claim 12, wherein:
the tubing and component management apparatus further includes a connection mechanism configured to maintain the second segment in the closed position;
wherein the connection mechanism is a mechanical lock or magnetic coupling.

16. The bioprocessing system of any one of claim 12, wherein:
the at least one mounting bracket is a plurality of mounting brackets;
wherein the plurality of mounting brackets include a plurality of filter heater mounting brackets configured for mounting filter heaters in a position generally above the frame; and
wherein the plurality of mounting brackets further include a plurality of pinch valve mounting brackets configured for receiving pinch valves, the plurality of pinch valve mounting brackets depending downwardly from the frame.

17. The bioprocessing system of any one of claim 12, wherein:
the frame has a peripheral shape that corresponds to an interior sidewall of the bioreactor vessel and internal baffles of the bioreactor vessel.

18. The bioprocessing system of any one of claim 12, wherein:
at least one of the segments is movable so as to extend from an access door opening in the bioreactor vessel.

19. The bioprocessing system of any one of claim 12, further comprising a filter heater, the filter heater comprising:
a sleeve having a plurality of interconnected sleeve elements, the sleeve having a top opening;
wherein the sleeve has a cross-sectional area that is greatest at the top opening and decreases moving away from the top opening; and
wherein the sleeve elements are configured so as to be biased outwardly upon insertion of a filter heater through the top opening.

20. The bioprocessing system of claim 19, wherein:
the plurality of sleeve elements are interconnected by webs of material adjacent to the top opening; and
wherein distal portions of the plurality of sleeve elements opposite the top opening are configured to be resiliently biased by the filter heater.
